(19) United States
(12) Patent Application Publication
(10) Pub. No.: US 2024/0233921 A1
(43) Pub. Date: Jul. 11, 2024

(54) COLLABORATIVE ARTIFICIAL INTELLIGENCE ANNOTATION PLATFORM LEVERAGING BLOCKCHAIN FOR MEDICAL IMAGING

(71) Applicant: The General Hospital Corporation, Boston, MA (US)

(72) Inventor: Synho Do, Boston, MA (US)

(21) Appl. No.: 18/559,016

(22) PCT Filed: May 4, 2022
(86) PCT No.: PCT/US2022/072103

§ 371 Date: Nov. 3, 2023

Related U.S. Application Data
(60) Provisional application No. 63/184,175, filed on May 4, 2021.

(51) Int. Cl. G16H 30/40

(52) U.S. Cl. CPC G16H 30/40

(57) ABSTRACT

A system and method are provided for providing a collaborative annotation platform. The method includes enabling access to a collaborative annotation project associated with at least one medical image. The method also includes receiving crowd-sourced annotations associated with the at least one medical image from a set of annotators. The method also includes evaluating the crowdsourced annotations and generating an annotation record associated with the at least one medical image based on the evaluation of the crowdsourced annotations.

### CROSS-REFERENCE TO RELATED APPLICATIONS

This application claims priority to U.S. Provisional Application No. 63/184,175 filed May 4, 2021, the entirety of which is incorporated herein by reference.

### STATEMENT REGARDING FEDERALLY SPONSORED RESEARCH

N/A.

### BACKGROUND

Medical imaging is a key tool in the practice of modern clinical medicine. Imaging is used in an extremely broad array of clinical situations, from diagnosis to delivery of therapeutics to guiding surgical procedures. While medical imaging provides an invaluable resource, it also consumes extensive resources. For example, imaging systems are expensive and are efficiently utilized when downtime is controlled. Furthermore, imaging systems require extensive human interaction to setup and operate, and then to analyze the images and make clinical decisions.

As the field of machine learning (“ML”) and artificial intelligence (“AI”) becomes more mature, researchers working on medical ML/AI aim to integrate more data to improve their models, as opposed to merely changing algorithm architecture. Ensuring high-quality annotations is critically important in medicine: imaging can be nondiagnostic and intra- and interobserver variability is high. As much as 25% of radiologists don't agree with other radiologists diagnoses and 30% don't agree with their own previous decisions. Ultimate ground truth, such as pathology reports, is not always available, and trained models often rely on “soft” annotated ground truth. Biases from poorly annotated datasets can result in negative consequences for ML algorithms in clinical use. However, to this day, few available collaborative annotation platforms for ML systems are capable of handling medical imaging.

### SUMMARY

The present disclosure provides systems and methods that reduce the total investment of human time required for medical imaging applications. In one non-limiting example, systems and methods are provided for facilitating the acceleration of the annotation process and development of medical imaging datasets.

The disclosure relates to platforms allowing medical entities, such as, e.g., researchers, commercial vendors, etc., to accelerate the annotation and development of medical imaging datasets. More specifically, embodiments enable labeling for classification and object detection tasks and provide various data and project management tools.

Improving the quality of database includes the participation of well-trained experts and a thorough curation process, which can be based on voluntary commitment. Crowdsourcing data collection methods can be easily contaminated by mislabeling caused by undertrained participants. For example, consider that the value of the data or accuracy of annotation may be easily estimated. In this scenario, it is possible to construct a high-quality dataset with an appropriate proportion of positive features for AI training, by exchanging or trading datasets between medical entities (e.g., researchers, vendors, etc.). Furthermore, this transaction can be fairly evaluated and securely monitored. Accordingly, embodiments described herein introduce a web-based, zero-footprint collaborative annotation tool for medical imaging data. As one non-limiting example, a proof of concept can include implementing the platform with pretrained AI models and blockchain features and using them to create preliminary annotations of a chest X-ray dataset for classification tasks.

Blockchain technology has been widely recognized to deliver decentralization and transparency to solutions in many areas. Some attempts have been made in medicine to utilize those benefits, mostly when handling electronic health records, promising better management for data ownership, sharing, or authorization. Nevertheless, only rarely attempts to utilize blockchain result in developing a tool useful in the clinical setting. Embodiments described herein explored how blockchain could encourage transparency and trust when crowdsourcing annotations are practiced by saving user activity in an immutable ledger. Blockchain technology has the advantage of defending against data manipulation without the installation of an additional security system. Embodiments described herein achieve security for image upload, annotation record modulation, etc, without compromising user convenience. Additionally, blockchain can give incentives for annotators (via, e.g., a blockchain currency or reward), inspire anonymous data sharing, etc.

In accordance with one aspect of the disclosure, a collaborative annotation system is provided that includes an electronic processor. The electronic processor is configured to enabling access to a collaborative annotation project associated with at least one medical image. The electronic processor is also configured to receive crowdsourced annotations associated with the at least one medical image from a set of annotators. The electronic processor is also configured to evaluate the crowdsourced annotations. The electronic processor is also configured to generate an annotation record associated with the at least one medical image based on the evaluation of the crowdsourced annotations.

In accordance with another aspect of the disclosure, a method is provided that provides a collaborative annotation platform. The method includes enabling, with an electronic processor, access to a collaborative annotation project associated with at least one medical image. The method also includes receiving, with the electronic processor, crowdsourced annotations associated with the at least one medical image from a set of annotators. The method also includes evaluating, with the electronic processor, the crowdsourced annotations. The method also includes generating, with the electronic processor, an annotation record associated with the at least one medical image based on the evaluation of the crowdsourced annotations.

In accordance with yet another aspect of the disclosure, a collaborative annotation system is provided that includes an electronic processor. The electronic processor is configured to define a collaborative annotation project associated with a set of medical images. The electronic processor is also configured to obtain crowdsourced annotations for the collaborative annotation project from a dispersed group of annotators. The electronic processor is also configured to evaluate the crowdsourced annotations. The electronic processor is also configured to generate at least one annotation record based on the evaluation of the crowdsourced annotations.

In accordance with yet another aspect of the disclosure, a collaborative annotation system is provided that includes an electronic processor. The electronic processor is configured to access at least one annotation record, wherein the at least one annotation record is based on crowdsourced annotations obtained for a collaborative annotation project associated with a set of medical images. The electronic processor is also configured to generate training data based on the at least one annotation record.

In accordance with yet another aspect of the disclosure, a collaborative annotation system is provided that includes an electronic processor. The electronic processor is configured to access training data associated with annotation records based on crowdsourced annotations obtained for a collaborative annotation project associated with a set of medical images. The electronic processor is also configured to develop a model using machine learning using the training data, wherein the model is associated with a medical image analysis function.

In accordance with yet another aspect of the disclosure, a collaborative annotation system is provided that includes an electronic processor. The electronic processor is configured to obtain crowdsourced annotations associated with at least one medical image from a dispersed group of annotators. The electronic processor is also configured to evaluate the crowdsourced annotations to determine an annotation contribution to the crowdsourced annotations for each annotator included in the dispersed group of annotators. The electronic processor is also configured to generate and associate a digital reward for at least one annotator included in the dispersed group of annotators based on a corresponding annotation contribution for the at least one annotator.

The foregoing and other aspects and advantages of the disclosed embodiments will appear from the following description. In the description, reference is made to the accompanying drawings which form a part hereof, and in which there is shown by way of illustration embodiments of the disclosed technology. Any such embodiment does not necessarily represent the full scope of the disclosed technology, however, and reference is made therefore to the claims and herein for interpreting the scope of the disclosed technology.

### BRIEF DESCRIPTION OF THE DRAWINGS

FIG. 1 is an example of a collaborative annotation system in accordance with the present disclosure.

FIG. 2 is an example of hardware that can be used to implement a server shown in FIG. 1 in accordance with the present disclosure.

FIG. 3 is a schematic illustration of user roles with respect to a collaborative annotation platform in accordance with the present disclosure.

FIG. 4 is an example of a data owner user interface for uploading medical images for use with the collaborative annotation platform in accordance with the present disclosure.

FIGS. 5-6 are examples of project manager user interfaces for managing a collaborative annotation project with the collaborative annotation platform in accordance with the present disclosure.

FIG. 7 illustrates an example dataflow between modularized functions provided by the collaborative annotation platform in accordance with the present disclosure.

FIG. 8 is an example medical image in accordance with the present disclosure.

FIG. 9 is an example interface associated with the collaborative annotation platform that illustrates annotations by category and associated confidence levels in accordance with the present disclosure.

FIG. 10 is an example graph illustrating a distribution of time spent annotating varied depending on both label type and radiologist in accordance with the present disclosure.

FIG. 11 is an example annotation evaluation sheet in accordance with the present disclosure.

FIG. 12 is an example process for determining a digital reward in accordance with the present disclosure.

FIG. 13 is a flowchart of an example method of providing a collaborative annotation platform using the system of FIG. 1 in accordance with the present disclosure.

### DETAILED DESCRIPTION

The present disclosure provides systems and methods that can reduce human and/or trained clinician time required to analyze medical images. As one non-limiting example, the present disclosure provides example of the inventive concepts provided herein applied to the analysis of x-rays, however, other imaging modalities beyond x-rays and applications within each modality are contemplated, such as echocardiograms, MRI, CT, PET, SPECT, optical, digital pathological images, and the like.

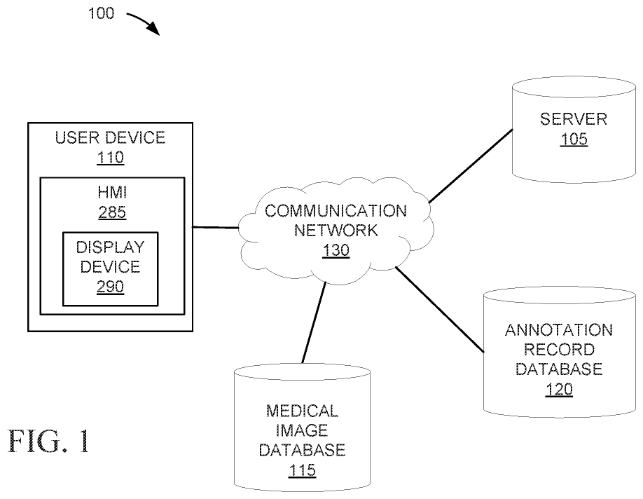

FIG. 1 schematically illustrates a system 100 for collaborative annotation of medical images according to some embodiments. In the illustrated example, the system 100 includes a server 105, a user device 110, a medical image database 115, and an annotation record database 120. In some embodiments, the system 100 includes fewer, additional, or different components in different configurations than illustrated in FIG. 1. As one example, the system 100 may include multiple servers 105, user devices 110, medical image databases 115, annotation record databases 120, or a combination thereof. As another example, one or more components of the system 100 may be combined into a single device, such as, e.g., the server 105 and medical image database 115 and/or the annotation record database 120 or the medical image database 115 and the annotation record database 120.

The server 105, the user device 110, the medical image database 115, and the annotation record database 120 communicate over one or more wired or wireless communication networks 130. Portions of the communication networks 130 may be implemented using a wide area network, such as the Internet, a local area network, such as a Bluetooth™ network or Wi-Fi, and combinations or derivatives thereof. Alternatively or in addition, in some embodiments, components of the system 100 communicate directly as compared to through the communication network 130. Also, in some embodiments, the components of the system 100 communicate through one or more intermediary devices not illustrated in FIG. 1.

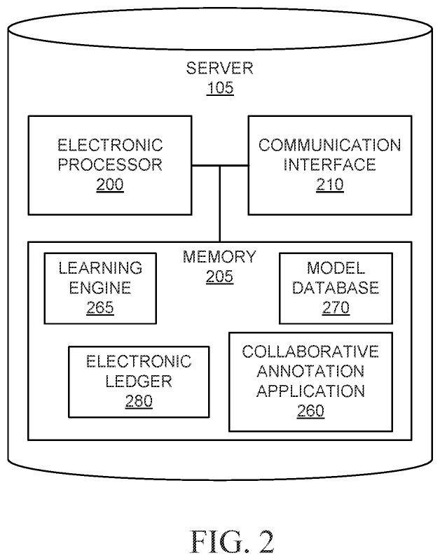

The server 105 is a computing device, such as a server, a database, or the like. As illustrated in FIG. 2, the server 105 includes an electronic processor 200, a memory 205, and a communication interface 210. The electronic processor 200, the memory 205, and the communication interface 210 communicate wirelessly, over one or more communication lines or buses, or a combination thereof. The server 105 may include additional components than those illustrated in FIG. 2 in various configurations. For example, the server 105 may also include one or more human machine interfaces, such as a keyboard, keypad, mouse, joystick, touchscreen, display device, printer, speaker, and the like, that receive input from a user, provide output to a user, or a combination thereof. The server 105 may also perform additional functionality other than the functionality described herein. Also, the functionality described herein as being performed by the server 105 may be distributed among multiple servers or devices (e.g., as part of a cloud service or cloud-computing environment), combined with another component of the system 100 (e.g., combined with the user device 110, another component(s) of the system 100, or the like), or a combination thereof.

The communication interface 210 may include a transceiver that communicates with the user device 110, the medical image database 115, the annotation record database 120, or a combination thereof over the communication network 130 and, optionally, one or more other communication networks or connections. The electronic processor 200 includes a microprocessor, an application-specific integrated circuit (“ASIC”), or another suitable electronic device for processing data, and the memory 205 includes a non-transitory, computer-readable storage medium.

The electronic processor 200 can access and execute computer-readable instructions (“software”) stored in the memory 205. The software may include firmware, one or more applications, program data, filters, rules, one or more program modules, and other executable instructions. For example, the software may include instructions and associated data for performing a set of functions, including the methods described herein.

For example, as illustrated in FIG. 2, the memory 205 includes a collaborative annotation application 260 (referred to herein as “the application 260”). The application 260 is a software application executable by the electronic processor 200 in the example illustrated and as specifically discussed below, although a similarly purposed module can be implemented in other ways in other examples. As described in more detail below; the electronic processor 200 executes the application 260 to provide a collaborative annotation platform. The collaborative annotation platform can facilitate the acceleration of medical imaging annotation and development of medical imaging datasets (e.g., as training data for one or more machine learning and/or artificial intelligence models). As one example, in some embodiments, the electronic processor 200 executes the application 260 to enable a collaborative annotation platform for performing crowdsourcing of annotation data, performs an evaluation of the crowdsourced annotation data, or a combination thereof. As one example, the application 260 (when executed by the electronic processor 200) may provide a web-based, zero-footprint collaborative annotation tool for medical imaging data that facilitates crowdsourcing annotations for medical imaging.

As also illustrated in FIG. 2, the memory 205 may store a learning engine 265 and a model database 270). In some embodiments, the learning engine 265 develops one or more models using one or more machine learning functions. Machine learning functions are generally functions that allow a computer application to learn without being explicitly programmed. In particular, the learning engine 265 is configured to develop an algorithm or model based on training data. For example, to perform supervised learning, the training data includes example inputs and corresponding desired (for example, actual) outputs, and the learning engine progressively develops a model (for example, a classification model, an object detection model, etc.) that maps inputs to the outputs included in the training data. Machine learning performed by the learning engine 265 may be performed using various types of methods and mechanisms including but not limited to decision tree learning, association rule learning, artificial neural networks, inductive logic programming, support vector machines, clustering. Bayesian networks, reinforcement learning, representation learning, similarity and metric learning, sparse dictionary learning, and genetic algorithms. These approaches allow the learning engine 265 to ingest, parse, and understand data and progressively refine models for data analytics, including medical imaging analytics and annotation.

As noted above, in some embodiments, the electronic processor 200 executes the application 260 to provide a collaborative annotation platform for performing crowdsourcing of annotation data, performs an evaluation of the crowdsourced annotation data, or a combination thereof. In some embodiments, the electronic processor 200 may use the crowdsourced annotation data (e.g., annotation records stored in the annotation record database 120) as training data for one or more of the models.

Models generated by the learning engine 265 can be stored in the model database 270). In some embodiments, the models generated by the learning engine 265 can perform medical image analysis functions, such as, e.g., predictions, probabilities, feature activation maps, etc. As one example, a model may include, e.g., a classification model, an object detection model, a segmentation model, another type medical imaging analysis model, and the like. As illustrated in FIG. 2, the model database 270) is included in the memory 205 of the server 105. It should be understood, however, that, in some embodiments, the model database 270 is included in a separate device accessible by the server 105 (included in the server 105 or external to the server 105).

As also illustrated in FIG. 2, the memory 250 may store an electronic ledger 280. The electronic ledger 280 can be, e.g., a type of database for storing information or data records, such as one or more records of annotation transactions (e.g., annotation records). In some embodiments, the electronic ledger 280 is part of a distributed ledger (or other database). A distributed ledger (or a shared ledger) is a type of database of digital data (transaction records) that is replicated, shared, and synchronized across each member (referred to as a node) of a network such that each member has its own private ledger. In general, a distributed ledger uses independent computing devices or servers for each node (or member) to record, share, and synchronize transactions in their respective electronic ledgers instead of keeping data centralized as in a traditional ledger. A blockchain is one type of distributed ledger but embodiments described herein are not limited to any particular type of distributed ledger.

Alternatively or in addition, in some embodiments, the electronic ledger 280 is a centralized ledger maintained by the server 105, as illustrated in FIG. 2. In some embodiments, a central ledger exists on a server (e.g., the server 105) that is part of a peer-to-peer network where the electronic ledgers 280 are replicated and agree. In some embodiments, the electronic ledgers 280 create (or generate) hashed information based on user activity (e.g., an annotation record or transaction). As one example, when a user annotates a medical image, user activity (i.e., the user's annotation of the medical image) can be generated as hashed information.

In some embodiments, proof of stake, or proof of work, may be used (also referred to herein as “proof of contribution”). Blocks may be created from these hashes and may be verified using a proof of work procedure before new data can be added to the electronic ledger 280. As blocks are added, the proof of work becomes more difficult as the nodes must process all of the previous blocks to add new blocks. Proof of work difficulty increases with increased blocks, nodes, and difficulty in verifying blocks. A server (e.g., the server 105) may accept blocks by creating the next block, those that don't agree are ignored, and only honest servers with duplicate electronic ledgers may be accepted. In some embodiments, servers may be mining computers (or another component of the system 100 may also provide the mining function) for the blockchain and provide proof of work or consensus. In some embodiments, conventional mining, staking pools, or the like may perform the mining, or tokenizing may be used.

Accordingly, in some embodiments, the electronic processor 200 can implement the electronic ledger 280 as a blockchain to track user activity, including, e.g., annotating images, uploading medical image data, exporting medical image data, and the like. Implementing a blockchain can facilitate better security and traceability of medical datasets, especially when considering global platforms that deal with sensitive data, such as medical imaging data. As described in greater detail below, in some embodiments, the blockchain (or blockchain data) may be used to calculate a value for a dataset (e.g., an annotation record) and estimate a reward (e.g., a digital reward or credit) for annotates via blockchain currency (e.g., a cryptocurrency) to facilitate, among other things, accurate annotation. Accordingly, implementation of blockchain technology can encourage transparency and trust when crowdsourcing annotations by saving user activity in an immutable ledger (e.g., as hashed information in the electronic ledger 280)

Returning to FIG. 1, the medical image database 115 stores medical imaging data. In some embodiments, the medical image database 115 is a picture archiving and communication system (“PACS”). Medical imaging data can include, e.g., a plurality of medical images. Alternatively or in addition, the medical imaging data can include metadata associated with a medical image (e.g., time-date stamp, medical technician information, patient identification information, etc.), medical records associated with a medical image (e.g., a radiological report), a clinical report or record associated with a patient associated with the medical image (e.g., a patient history), and the like. Accordingly, the medical image database 115 may store images, reports, test results, notes, and other medically related data. As one example, in some embodiments, the medical image database 115 stores a set of digital imaging and communication in medicine (“DICOM”) files. As noted above, in some embodiments, the medical image database 115 may be distributed among multiple devices, such as, e.g., multiple databases. Alternatively or in addition, the medical image database 115 may be combined with another device, such as, e.g., the annotation record database 120, the server 105, the user device 110, another component of the system 100, or a combination thereof.

The annotation record database 120 stores one or more annotation records associated with medical imaging data (e.g., crowdsourced training data). An annotation record can include, e.g., one or more annotations (or other user activity) associated with one or more medical images (e.g., a medical image included in the medical imaging data of the medical image database 115). An annotation record may be associated with a single medical image. As one example, when a first annotator and a second annotator annotate the same medical image, the annotation record for that medical image can include the annotations provided by the first annotator and the annotations provided by the second annotator. Alternatively or in addition, in some embodiments, an annotation record may be associated with multiple medical images, where each medical image shares at least one characteristic or parameter (e.g., a type of medical image, an imaging modality or system used to capture the medical image, a viewpoint of the medical image, etc.). As one example, a first annotator annotates a first chest x-ray and a second chest x-ray and a second annotator annotates the first chest x-ray and a third chest x-ray. Following this example, the annotation record may include the first annotators annotations of the first chest x-ray and the second chest x-ray and the second annotators annotations of the first chest x-ray and the third chest x-ray. Alternatively or in addition, an annotation record can be associated with an annotator. As one example, a first annotation record can be associated with user activity with a first medical image of a first annotator and a second annotation record can be associated with user activity with a second medical image of a first annotator. As yet another example, a first annotation record can be associated with user activity with a first medical image of a first annotator and a second annotation record can be associated with user activity with a second medical image of a second annotator.

As noted above, in some embodiments, the annotation record database 120 may be distributed among multiple devices, such as, e.g., multiple databases. Alternatively or in addition, the annotation record database 120 may be combined with another device, such as, e.g., the medical image database 115, the server 105, the user device 110, another component of the system 100, or a combination thereof. As noted above, in some embodiments, the electronic processor 200 may use crowdsourced annotation data (e.g., one or more annotation records) as training data for one or more of the models stored in the model database 270).

The user device 110 can include a computing device, such as a desktop computer, a laptop computer, a tablet computer, a terminal, a smart telephone, a smart television, a smart wearable, or another suitable computing device that interfaces with a user. Although not illustrated in FIG. 1, the user device 110 may include similar components as the server 105, such as electronic processor (e.g., a microprocessor, an application-specific integrated circuit (“ASIC”), or another suitable electronic device), a memory (e.g., a non-transitory, computer-readable storage medium), a communication interface, such as a transceiver, for communicating over the communication network 130 and, optionally, one or more additional communication networks or connections. As one example, to communicate with the server 105 (or another component of the system 100), the user device 110 may store a browser application or a dedicated software application executable by an electronic processor. The system 100 is described herein as providing, among other things, a collaborative annotation platform or service for medical images through the server 105. However, in other embodiments, the functionality described herein as being performed by the server 105 may be locally performed by the user device 110. For example, in some embodiments, the user device 110 may store the application 260, the learning engine 265, the model database 270), the electronic ledger 280, or a combination thereof.

In the illustrated example of FIG. 1, the user device 110 may also include a human-machine interface 285 for interacting with a user. The human-machine interface 285 may include one or more input devices, one or more output devices, or a combination thereof. Accordingly, in some embodiments, the human-machine interface 285 allows a user to interact with (e.g., provide input to and receive output from) the user device 110. For example, the human-machine interface 285 may include a keyboard, a cursor-control device (e.g., a mouse), a touch screen, a scroll ball, a mechanical button, a display device (e.g., a liquid crystal display (“LCD”)), a printer, a speaker, a microphone, or a combination thereof. As illustrated in FIG. 1, in some embodiments, the human-machine interface 285 includes a display device 290. The display device 290 may be included in the same housing as the user device 110 or may communicate with the user device 110 over one or more wired or wireless connections. As one example, in some embodiments, the display device 290 is a touchscreen included in a laptop computer or a tablet computer. In other embodiments, the display device 290 is a monitor, a television, or a projector coupled to a terminal, desktop computer, or the like via one or more cables.

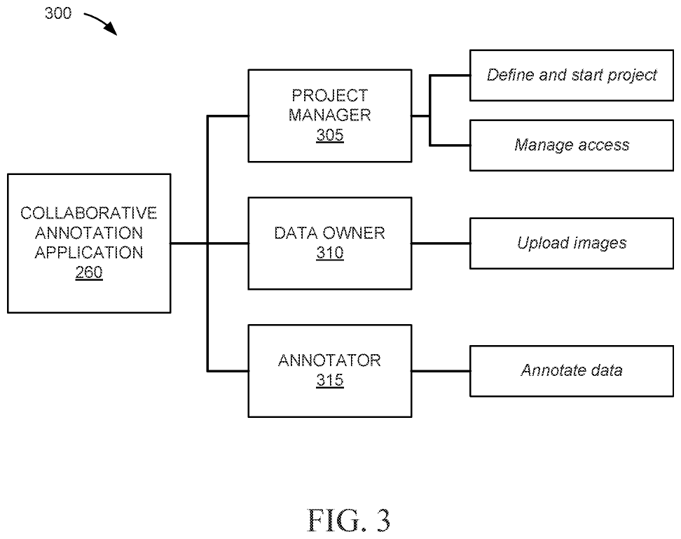

A user may use the user device 110 to interact with the collaborative annotation platform. FIG. 3 schematically illustrates example diagram 300 of user roles (or stakeholders) of the collaborative annotation platform in accordance with some embodiments. As illustrated in FIG. 3, example users may include a data owner 305, a project manager 310, and an annotator 315.

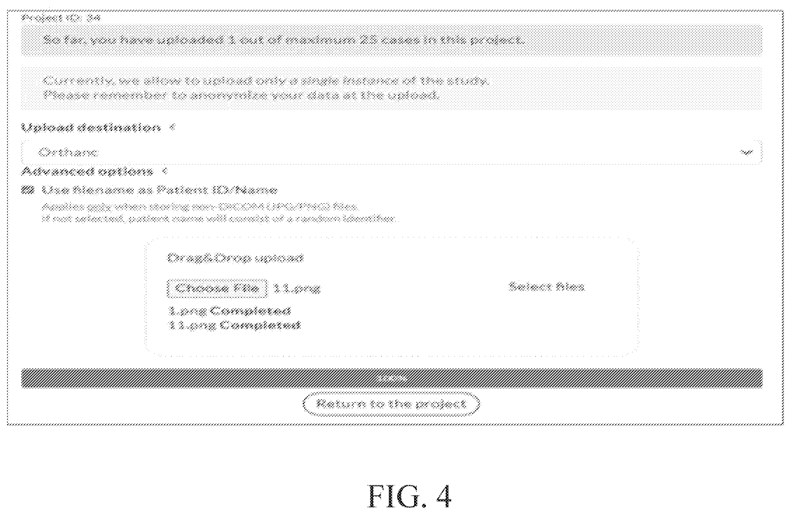

The data owner 310 can include a healthcare entity, group, or organization that manages and maintains medical data (e.g., a hospital, a healthcare clinic, an urgent care clinic, etc.). A data owner 310 can use the user device 110 to upload (or otherwise enable access to) a set of medical images (e.g., as a collaborative annotation project). As one example, as illustrated in FIG. 4, data owners 310 can upload images with additional options for choosing desired data storage systems and file naming conventions.

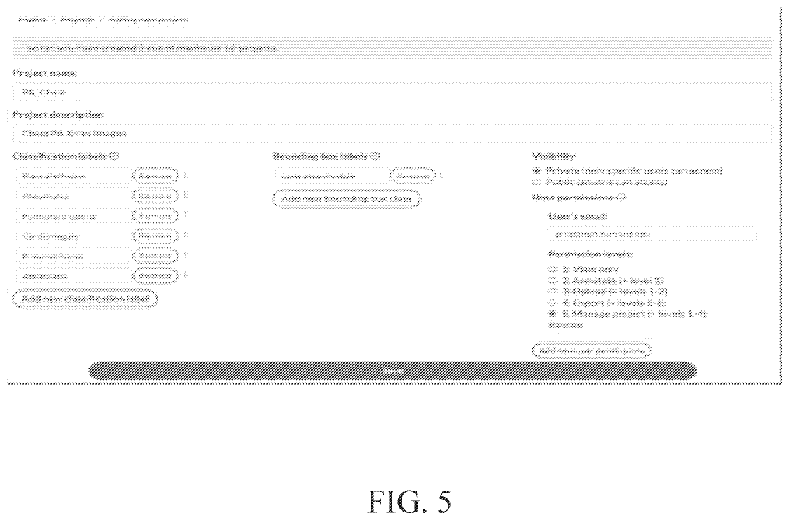

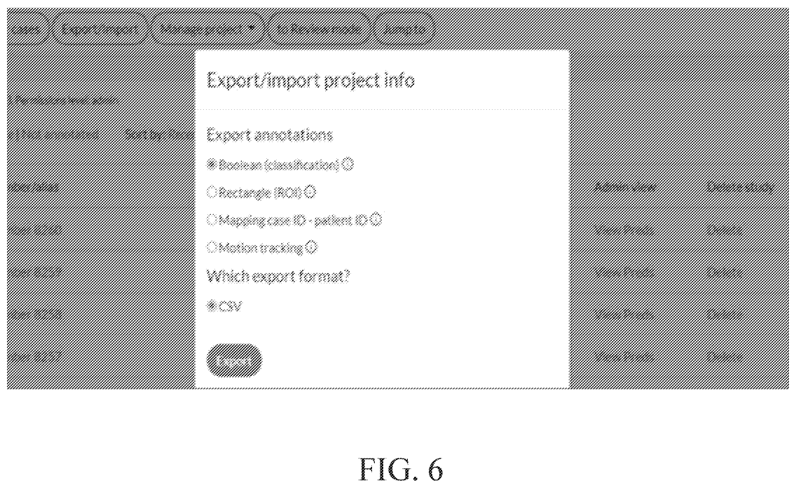

The project manager 305 may use the user device 110 to define a collaborative annotation project, start a collaborative annotation project, manage access to a collaborative annotation project, and the like (based on the medical images provided by the data owner 305). In some embodiments, the project manager 305 is a member, associated with, or otherwise affiliated with the same healthcare entity, group, or organization of the data owner 310. For example, stored studies can be organized privately by users into projects (e.g., one or more collaborative annotation projects). The collaborative annotation platform can allow project managers 305 to assign access privileges to a collaborative annotation project for other users, such as, e.g., other readers, annotators, or the like, which prevents unwanted access to sensitive medical data. Specific users can have various access levels, limiting some features, such as data export, progress tracking, project statistics, and management. As one example, as illustrated in FIG. 5, a project manager 305 can coordinate projects by specifying labels as in accordance with planned AI tasks, controlling visibility for all users, granting and revoking permissions for annotators, and the like. Alternatively or in addition, a project manager 305 can export project-related data, including annotations by all team members and information about time spent on labeling by users, as illustrated in FIG. 6.

The annotator 315 can use the user device 110 to access and annotate a collaborative annotation project (e.g., a set of medical images designated or selected for crowdsourcing annotation). An annotation record is generated based on the annotator's 315 interaction with the collaborative annotation project (or a medical image included therein). For example, all annotations (or user activity) are monitored and stored as an annotation record (e.g., in the annotation record database 120). An annotation record may include information on time spent on a single case (e.g., a collaborative annotation project or one or more medical images included therein) from the moment the collaborative annotation project (or medical image(s) therein) is completely loaded to the click of the submit button, the time of mouse clicking for labeling, or motionless duration to evaluate each label's duration of tasks.

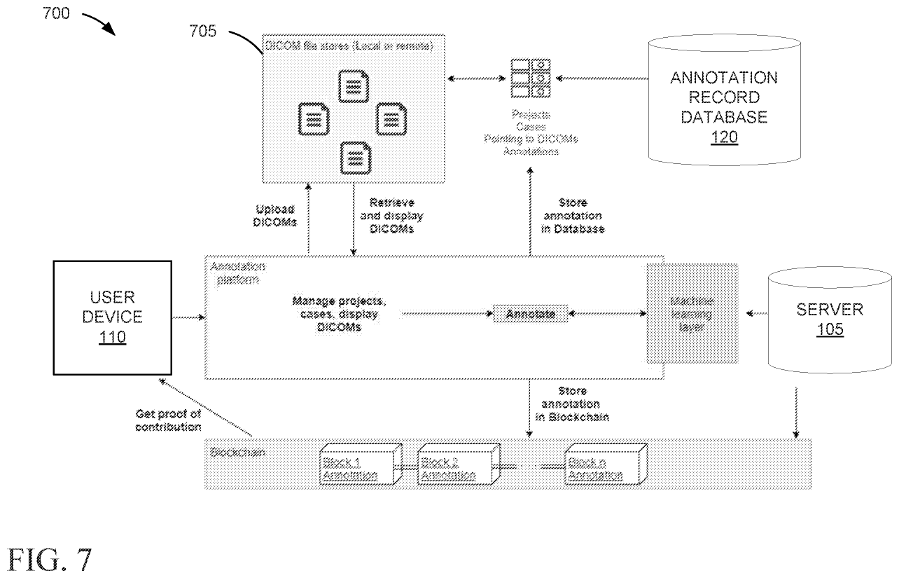

FIG. 7 illustrates an example implementation of the collaborative annotation platform 700 provided by the system 100 of FIG. 1 according to some embodiments. FIG. 7 illustrates an example dataflow between modularized functions provided by the collaborative annotation platform 700.

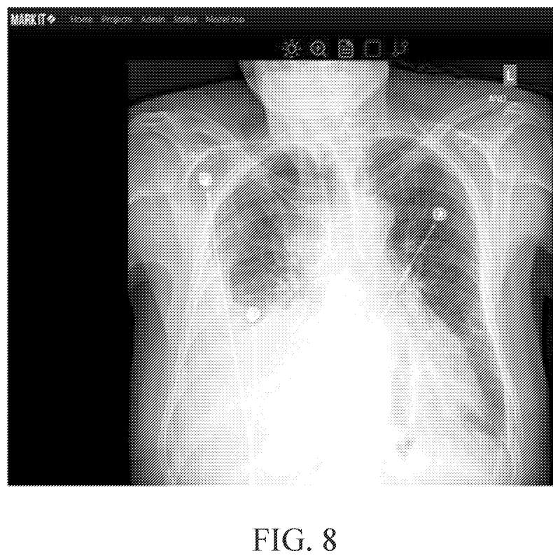

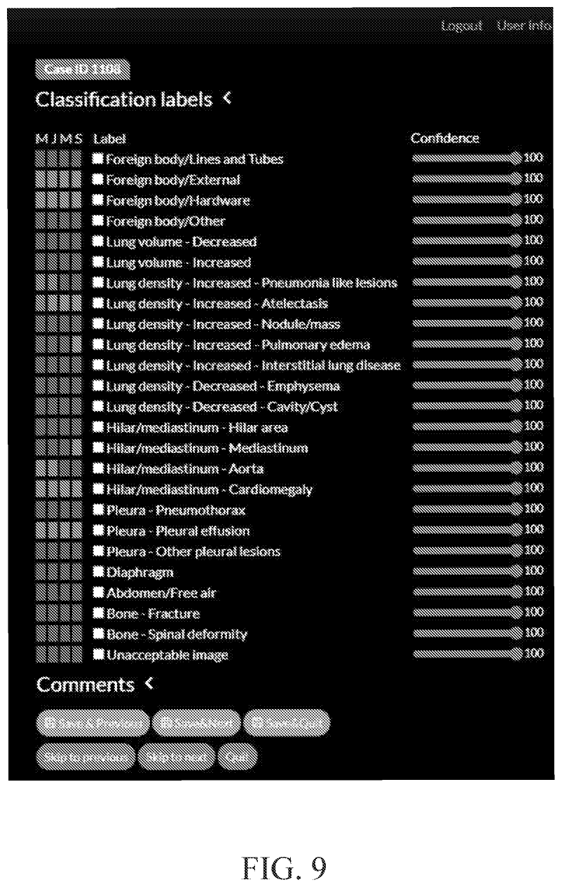

As illustrated in FIG. 7, the collaborative annotation platform 700 includes an image annotation module 705 for annotating one or more medical images. The image annotation module 705 can include a DICOM view, one or more labeling or annotation tools, etc. As one example, the DICOM viewer allows annotators to change image brightness and contrast and to zoom-in to read images in full resolution. As one example, FIG. 8 illustrates an example medical image displayed within a DICOM view. For classification, the collaborative annotation platform 700 (via the image annotation module 705) provides the ability to save Boolean annotations and associated confidence levels as six iterative grades (0, 20, 40, 60, 80, and 100% confidence), as illustrated in FIG. 9. For object detection, rectangular or free-line region of interest (“ROI”) annotations can be available. Users can define their subcategories of labels for the specific target projects when they set up a collaborative annotation project. This can be modified if the user is either an owner or manager of the project (e.g., the project manager 305 and/or the data owner 310).

Accordingly, the collaborative annotation platform can fetch (or access) medical images from a vendor-neutral DICOM storage (e.g., the medical image database 115). As one example, the collaborative annotation platform may be implemented via a connection with both standard PACS systems and DICOM web-based RESTful web services and application programming interfaces (“APIs”).

As one proof of concept, Orthanc [https://www.orthanc-server.com, Liege, Belgium] and Google DICOM Store (through Google Healthcare API, CA, USA) can be utilized, where image retrieval can be performed through WADO (Web Access to DICOMR: Persistent Objects) protocols. Connecting to standard PACS systems and fetching images with the C-GET protocol can also be implemented. Additionally, the collaborative annotation platform allows users to use non-DICOM image files, common in large-scale non-volumetric medical datasets (e.g., National Institutes of Health (“NIH”) and Stanford chest X-ray datasets). Within the collaborative annotation platform, patient information can be anonymized.

Users can export annotations in comma-separated values (“CSV”) format (e.g., for classification results, ROI labels, or a combination thereof). It is also possible to import radiological reports to the collaborative annotation platform, matching each radiological report with specific cases. The collaborative annotation platform is developed to optimize annotation workflow, especially in large-scale datasets with multiple collaborators and stakeholders, where the collaborative annotation platform takes into account each user's role, as illustrated in FIG. 3.

The collaborative annotation platform can be connected with a locally developed AI inference RESTful (Representational Status Transfer) service, running on the same device (e.g., the server 105) through a Docker container [https://www.docker.com, CA, USA]. This service includes four AI classification models for chest X-ray data, predicting: view position (i.e., AP: anterior-posterior vs. PA: posterior-anterior), pathologic features, gender, and age. View position and gender predictions are framed as binary classification tasks, feature prediction as multilabel classification and age prediction as a regression task. The collaborative annotation platform can further expand the collection of available pre-trained models and improve the performance of current models by changing datasets or model architectures.

A user of the collaborative annotation platform can request model prediction on the loaded image in real-time (or near real-time) by passing the input through a GPU-accelerated inference service. Images are sent to the service from DICOM storage via an API that evaluates sent data and returns predictions, probabilities, and feature activation maps.

Predictions are returned to users via one or more user interfaces. Feature activation maps in a form of gradient-weighted class activation mapping (“Grad-CAM”) can be overlaid over a DICOM image as a Red-Green-Blue-Alpha (“RGBA”) matrix with adjustable opacity.

The collaborative annotation platform can provide a “review mode” to evaluate discrepancies between annotators. The process of annotating medical imaging for machine learning purposes can be different from making diagnosis in the actual clinical environment, and annotators may have different standards for determining whether a particular feature exists or not. Therefore, the collaborative annotation platform can resolve disagreements in order to maintain consistency of the dataset. Project managers may save time by running smaller sample projects before the main annotation project to assess the presence of various problems. As one example, the collaborative annotation platform may implement this function by illustrating the annotators' labeling results and reliability in the form of a graphical representation, such as, e.g., a heatmap. Accordingly, the collaborative annotation platform may allow the second annotators to check the results agreements between preceding annotators. With this mode, annotators can develop better annotating strategies and prevent trial and error in main annotation projects.

Furthermore, this mode can be implemented for training or education purposes. For example, before implementing the main project, the collaborative annotation platform can implement a combined function that reduces unnecessary mistakes and pre-training sessions using the review mode. Users can quickly check their discordance in pretraining sessions within the collaborative annotation platform through review mode, and they can resolve the discordance problem.

Additionally, the collaborative annotation platform can include a blockchain implementation (e.g., the electronic ledger 280) to partially track user activity, including annotating images, uploading and exporting data, etc. As described above, blockchain can facilitate better security and traceability of medical datasets, especially when considering global platforms that deal with sensitive data, such as medical imaging.

### Experiments and Results

Three fellowship-trained radiologists classified the chest X-ray images as proof of concept in the private project. One thousand anonymized PA-view chest X-ray images with DICOM format of Massachusetts general hospital were uploaded to the collaborative annotation platform. Twenty-five classification labels were determined and assigned to the project (e.g., as illustrated in FIG. 6). The binary classification results were compared with AI-generated prediction results from in-house data, generated the time statistics, and estimated the task difficulty.

To suggest a clear analysis method, we only concentrated on seven critical labels with clinically high value (i.e., interstitial lung disease, pneumonia, pulmonary edema, pleural effusion, cardiomegaly, pneumothorax, and atelectasis). Other features can be analyzed in the same way and have similar characteristics.

The inter-rater agreement between the three annotators was measured (=0.90) based on all three annotator's results were matched, and we also measured Fleiss's Kappa value (=0.63) to assess the reliability of agreement between three raters when assigning categorical ratings in this case, seven pathological feature annotations. Among 7,000 labels that were annotated in total (7 labels times 1,000 images), 370 were labeled by all annotators as positive, and 5,954 as negative.

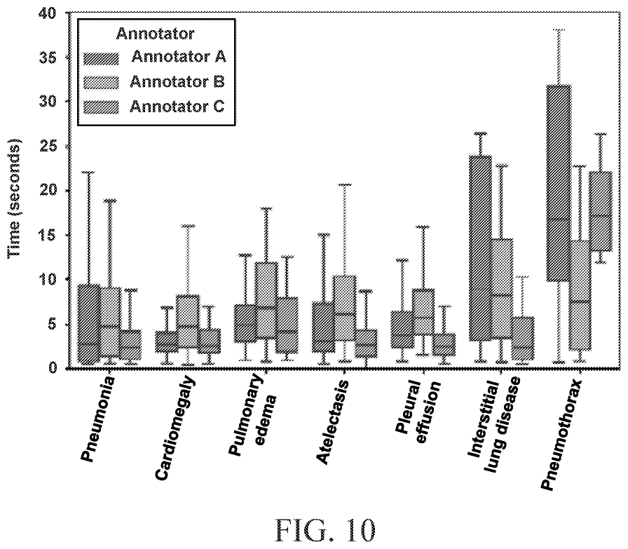

The remaining 676 labels had differences in assessments between readers. The 95% confidence interval of total mean labeling time was 6.16±0.21 seconds, and the cardiomegaly took the shortest labeling time (4.63±0.54 seconds): in contrast, the pneumothorax has the longest labeling time (13.92±3.93 seconds). As illustrated in FIG. 10, distribution of time spent annotating varied depending on both label type and radiologist.

When annotations on a new dataset are received, it can be important to understand the following: (1) How much is the data worth? (2) How much is any annotation worth? (3) Which annotator contributed and how much? For that, we formulate the value of the data based on the dataset characteristics, time cost of entering the annotation, and its annotation accuracy. The average labeling time was identified as an indicator for estimating the labor in the annotation. To calculate accuracy, we measured agreement between annotated label and pseudo-ground truth, defined as the majority rule between annotators.

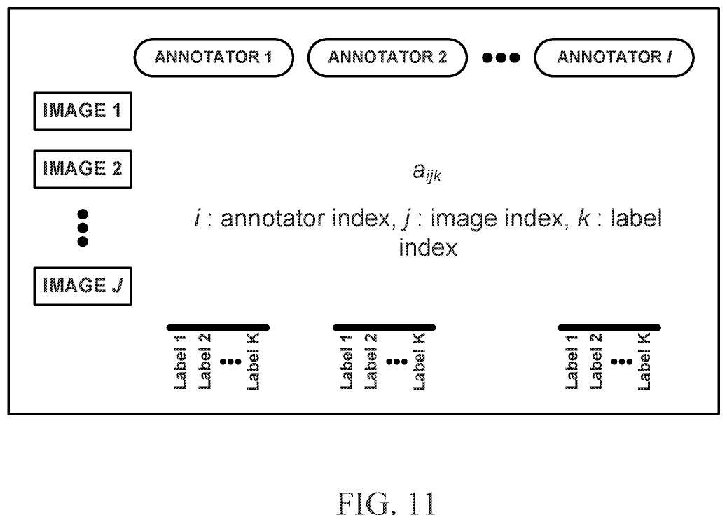

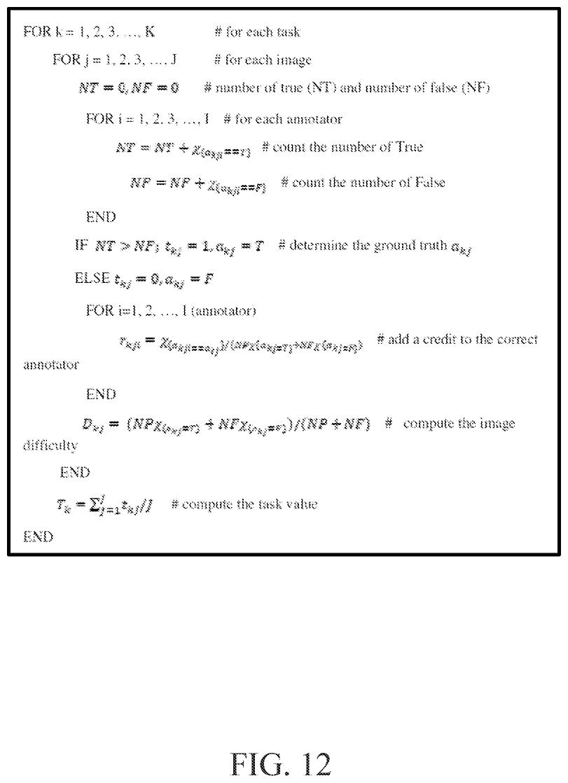

In order to evaluate the annotator's contribution for CXR PA dataset, we exported the binary classification data and generated the annotation evaluation sheet consisting of True or False. As illustrated in FIG. 11, for the k-th label of j-th image, the i-th annotator's annotation results (True or False) is recorded as aᵢⱼₖ. We consider the seven labels (e.g., as illustrated in FIG. 11) for 1000 cases annotated by three annotators, so I=3, J=1000, K=7. Using the table, we devise an algorithm to estimate each annotators' contribution, level of challenge of each image and task, respectively, and evaluate each annotator's reward (see Equation 1 below). FIG. 12 illustrates an example algorithm for calculating reward factors according to some embodiments.

With reference to the algorithm illustrated in FIG. 12, we evaluate the contribution credit of i-th annotator for k-th label of j-th image rₖⱼᵢ, the value of k-th label of j-th image for data Dₖⱼ, and the task value Tₖ. For example, the lower the correct answer rate, the higher the value of the image and task and the annotators' contribution. We set the pseudo answer for k-th label of j-th image and consider it as a ground truth for each task (k-th label of j-th image). To count the laboring factor, we put the normalized time mean for k-th label Fₖ. Here, χ_{{condition}} is a characteristic function having the value 1 if the condition is true, otherwise 0).

It is obvious that χ∈[a, b] can be normalized oppositely (b maps to −1, a maps to 1) as

$$\frac{2}{a - b} ( \frac{a + b}{z} - x ) ϵ [ - 1 , 1 ] .$$

Applying it on Dₖⱼ∈[0, 1] and rₖⱼᵢ∈[0, 0.5], we scale them as follows:

$${\tilde{D}}_{k j} = 2 * ( 0.5 - {D}_{k j} ) ϵ [ - 1 , 1 ] \\ {\tilde{r}}_{k j i} = 4 * ( 0.25 - {r}_{k j i} ) ϵ [ - 1 , 1 ]$$

Each annotator's reward reward(i) can be formulated as a linear combination of each factor as:

$$reward ( i ) = ( Total Budget ) * {\sum }_{k = 1}^{K} ( {T}_{k} + {\tilde{t}}_{k} ) {\sum }_{j = 1}^{J} {\tilde{D}}_{k j} {\tilde{r}}_{k j i} / {\sum }_{i = 1}^{I} {\sum }_{k = 1}^{K} ( {T}_{k} + {\tilde{t}}_{k} ) {\sum }_{j = 1}^{J} {\tilde{D}}_{k j} {\tilde{r}}_{k j i} \qquad ( 1 )$$

This approach considers the information on the dataset and the estimated annotation quality and the time it takes to determine it, and the label-specific accuracy of the annotator.

For our experiment, we assumed the value of the entire dataset as 1000T MED Token (a cryptocurrency used in the current research) for the seed money of the data trading system and distributed the collaborative annotation platform currency to three annotators (i.e., Annotator A: 371T, Annotator B: 347T, and Annotator C: 282T MED Token) from Equation 1 (above). Although 1000T MED Token was implemented, another cryptocurrency can be implemented within the collaborative annotation platform.

Equation 1 can be modified in different ways depending on the situation. As one example, the importance of various factors can be considered through the sum of weights. In reality, the value of data will change. Initially, we will start with a small amount of data, and the performance of artificial intelligence developed using this data will also have limitations. However, as a large amount of data is added gradually and more annotators label the data, the data's value will increase, and the entire data's value will increase. In this case, what is calculated by Equation 1 is repeated according to the change in the quantity and quality of the data, and the value will change accordingly.

To prevent non-expert annotators from exceeding experts in number making wrong ground truth, we introduced the AI as a quality controller. According to the AI result, we set a temporary ground truth and assumed that it has better performance than a random choice (i.e., coin tossing). We calculated Cohen's kappa values between AI and each annotator. If this value was greater than 0.05, we assumed that this annotator has better prediction power than random selection. In the example, all annotators have better performance than the threshold in each label, so we used all labels for the reward calculations.

We tested a Panacea blockchain in our implementation, which is developed on top of Cosmos SDK and Tendermint framework, but the collaborative annotation platform can be integrated into any framework that supports blockchain implementations. Interacting with the blockchain can be executed through the RESTful API and the command-line interface (CLI) for a Go (programming language) application. In our experiments, we were saving user activity as hashed information in separate transactions on the blockchain. Using this information, we tried to calculate the dataset's value and estimated the awards for annotators via blockchain currency to facilitate accurate annotation. In addition to this, you will be able to add more various applications and services.

### DISCUSSION

In the medical imaging field, annotation tasks require a trained radiologist's expertise, and even for simple tasks, crowdsourced annotations can be noisy or inaccurate. A successful crowdsourcing platform's benefits include, e.g., faster production of high-quality labeled datasets, more economical cost of obtaining annotations on the large datasets, and accelerated development of ML/AI for multiple medical imaging tasks.

The collaborative annotation platform described herein allows researchers and commercial vendors to accelerate the annotation and development of medical imaging datasets.

Our study confirms that quick annotation of large-scale images is possible in the above-mentioned platform. Results show high variability of the annotation speed between readers, which may help determine annotator engagement in the process.

Accordingly, embodiments described herein provide the development of a zero-footprint, web-based tool that is easy to implement on both local and global scale. Embodiments described herein suggest using a minimal number of features for the user-friendliness of the interface. Furthermore, annotators can be provided with additional clinical information, including, e.g., radiological reports or patient history in order to improve annotation accuracy as an option. High-reliability annotation results can be obtained by providing annotators with information comparable to the real clinical environment. To combat the widely known problem of “soft ground truth,” imaging may not always correlate with hidden clinical information.

The embodiments described herein attempt to overcome previous research limitations, presenting a robust platform dedicated to CNN-based ML/AI research in medical imaging. Connecting multiple RESTful services allows this platform to scale and rapidly increase further functionality.

As noted above, in some embodiments, the collaborative annotation platform supports classification and object detection. However, the collaborative annotation platform may support additional, different, or fewer medical imaging tools or functions. As one example, the collaborative annotation platform may support image segmentation, such as, e.g., a three-dimensional (“3D”) Slicer. Additionally, the collaborative annotation platform can support various medical imaging data and data types, including, e.g., volumetric images, non-image DICOM instances, such as DICOM-SEG or DICOM-SR, HISTI, NRRD, and the like.

As noted above, blockchain will enable us to obtain a free and fair data exchange system among researchers. Currently, most of the data is owned by healthcare providers, major national research institutes, and large research institutes. Ultimately, sharing all data without condition will help create new value. Still, it won't be easy to actively share data without compensation for intellectual labor, such as annotation and curation, reflecting the benefits of the institution that owns it. Accordingly, the collaborative annotation platform described herein can leverage blockchain's potential to reflect the value of the data and use it as currency for data transactions (e.g., the exchange of data among researchers). The issuance of currencies with a specific purpose for data exchange may also help as a means of data acquisition for artificial intelligence development while being less likely to cause ethical problems related to data ownership issues.

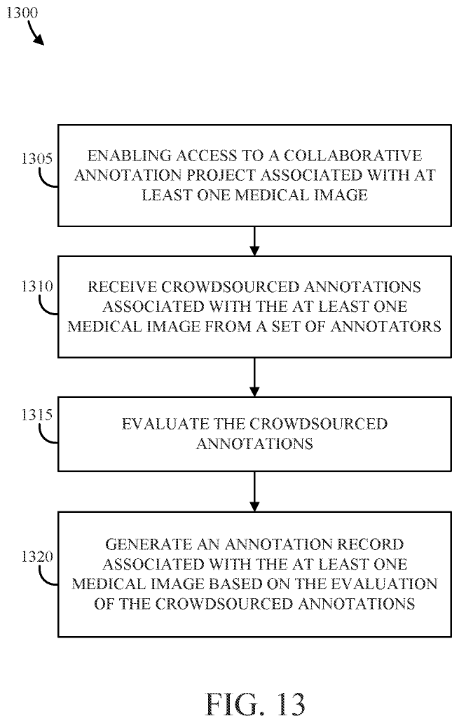

FIG. 13 is a flowchart of a method 1300 of providing a collaborative annotation platform according to some embodiments. The method 1300 is described as being performed by the server 105 (e.g., the electronic processor 200 executing the application 260). However, as noted above, the functionality described with respect to the method 1300 may be performed by other devices, such as the user device 110, or distributed among a plurality of devices, such as a plurality of servers included in a cloud service.

As illustrated in FIG. 13, the method 1300 includes enabling access to a collaborative annotation project associated with at least one medical image (at block 1305). As noted above, a collaborative annotation project may be generated or defined by a project manager 310 and/or a data owner 305, as described in greater detail above. As one example, a project manager 310) (or another user) can define a collaborative annotation project, start a collaborative annotation project, manage access to a collaborative annotation project, or the like by interacting (via, e.g., the user device 110) with the collaborative annotation platform (e.g., the application 260).

After generating or defining a collaborative annotation project, the user may request (or invite) one or more annotators to interact with the collaborative annotation project (including the one or more medical images included therein). Accordingly, in some embodiments, the electronic processor 200 may generate and transmit one or more requests for annotations (e.g., annotation data) from one or more annotators (e.g., annotators specified by the project manager 310). The one or more requests may be transmitted over the communication network 130 to a user device associated with an annotator (e.g., the user device 110). In response to receiving the request, the annotator may annotate the at least one medical image.

The electronic processor 200 can receive crowdsourced annotations associated with the at least one medical image from a set of annotators (at block 1310). The crowdsourced annotations may include annotation data from a dispersed group of annotators. The crowdsourced annotations may include annotation data (e.g., user activity with the at least one medical image) provided by each annotator included in the set of annotators. Accordingly, the crowdsourced annotations may include, e.g., a set of classification labels, a set of object detection labels, other annotation related data, etc. As noted above, in some embodiments, annotators may indicate a confidence level or metric for each annotation they provide. Accordingly, in such embodiments, the crowdsourced annotations may include a set of confidence metrics indicating a confidence level of a corresponding annotator with an associated crowdsourced annotation.

In response to receiving the crowdsourced annotations (at block 1310), the electronic processor 200 may evaluate the crowdsourced annotations (at block 1315). As described in greater detail above, the electronic processor 200 may evaluate the crowdsourced annotations to determine a value associated with the annotation data, a contribution of the annotator, an accuracy of the annotation data, etc.

In some embodiments, the electronic processor 200 determines a digital reward for each annotator based on the evaluation of the crowdsourced annotations, as described in greater detail above. In some embodiments, the electronic processor 200 determines the digital reward as a blockchain cryptocurrency.

After evaluating the crowdsourced annotations (at block 1315), the electronic processor 200 can generate an annotation record based on the evaluation of the crowdsourced annotations (at block 1320). As described in greater detail above, the annotation record can be used as training data by the learning engine 265 for training one or more machine learning and/or artificial intelligence models associated with performing a medical imaging function. In some embodiments, the electronic processor 200 stores the annotation record. The electronic processor 200 can store the annotation record locally (e.g., in the memory 205), such as, e.g., in the electronic ledger 280) (as described in greater detail above). Alternatively or in addition, the electronic processor 200 can store the annotation record remotely, such as, e.g., in the annotation record database 120.

Thus, the present disclosure provides systems and methods for providing a collaborative artificial intelligence annotation platform leveraging blockchain for medical imaging research.

The present technology has been described in terms of one or more preferred embodiments, and it should be appreciated that many equivalents, alternatives, variations, and modifications, aside from those expressly stated, are possible and within the scope of the invention.

1. A collaborative annotation system, the collaborative annotation system comprising:
an electronic processor configured to:
enable access to a collaborative annotation project associated with at least one medical image,
receive crowdsourced annotations associated with the at least one medical image from a set of annotators,
evaluate the crowdsourced annotations, and
generate an annotation record associated with the at least one medical image based on the evaluation of the crowdsourced annotations.

2. The system of claim 1, wherein the electronic processor is configured to generate and transmit a request for annotations to each annotator associated with the collaborative annotation project, wherein the set of annotators is associated with the collaborative annotation project.

3. The system of claim 1, wherein the crowdsourced annotations includes a set of classification labels associated with the at least one medical image, wherein each classification label is associated with an annotator included in the set of annotators.

4. The system of claim 1, wherein the crowdsourced annotations includes a set of object detection labels associated with the at least one medical image, wherein each object detection label is associated with an annotator included in the set of annotators.

5. The system of claim 1, wherein each crowdsourced annotation is associated with a confidence metric indicating a confidence level of a corresponding annotator with an associated crowdsourced annotation.

6. The system of claim 1, wherein the electronic processor is configured to determine a digital reward for each annotator based on the evaluation of the crowdsourced annotations.

7. The system of claim 6, wherein the digital reward is a blockchain cryptocurrency.

8. The system of claim 6, wherein the electronic processor is configured to evaluate the crowdsourced annotations by determining a contribution of each annotator to the crowdsourced annotations.

9. The system of claim 8, wherein the electronic processor is configured to determine the contribution of each annotator to the crowdsourced annotations by, for each annotator, determining a value associated with at least one crowdsourced annotation included in the crowdsourced annotation, wherein the at least one crowdsourced annotation is associated with an annotator included in the set of annotators.

10. The system of claim 9, wherein the electronic processor is configured to determine the value based on at least one of a characteristic of the at least one crowdsourced annotation, a time metric of entering the at least one crowdsourced annotation, and an accuracy of the at least one crowdsourced annotation.

11. The system of claim 1, wherein the electronic processor is configured to store the crowdsourced annotations to an electronic ledger.

12. The system of claim 11, wherein the electronic ledger is a blockchain.

13. The system of claim 1, wherein the electronic processor is configured to store the crowdsourced annotations as training data for at least one machine learning model associated with medical image analysis.

14. The system of claim 13, wherein the at least one machine learning model is a classification model.

15. The system of claim 13, wherein the at least one machine learning model is an object detection model.

16. A method of providing a collaborative annotation platform, the method comprising:
enabling, with an electronic processor, access to a collaborative annotation project associated with at least one medical image;
receiving, with the electronic processor, crowdsourced annotations associated with the at least one medical image from a set of annotators;
evaluating, with the electronic processor, the crowdsourced annotations; and
generating, with the electronic processor, an annotation record associated with the at least one medical image based on the evaluation of the crowdsourced annotations.

17. The method of claim 16, further comprising:
generating training data based on the annotation record;
developing a machine learning model using the training data; and
storing the machine learning model.

18. The method of claim 17, further comprising:
receiving a first medical image;
applying the machine learning model to the first medical image; and
generate a second medical image based on the application of the machine learning model, wherein the second medical image includes a predicted annotation for the first medical image.

19. The method of claim 16, further comprising:
as part of evaluating the crowdsourced annotations,
determining the contribution of each annotator to the crowdsourced annotations by
for each annotator,
determining a value associated with at least one crowdsourced annotation included in the crowdsourced annotation, wherein the at least one crowdsourced annotation is associated with an annotator included in the set of annotators.

20. The method of claim 19, wherein determining the value includes determining the value is based on at least one of a characteristic of the at least one crowdsourced annotation, a time metric of entering the at least one crowdsourced annotation, and an accuracy of the at least one crowdsourced annotation.

21. A collaborative annotation system, the system comprising:
an electronic processor configured to:
define a collaborative annotation project associated with a set of medical images,
obtain crowdsourced annotations for the collaborative annotation project from a dispersed group of annotators,
evaluate the crowdsourced annotations, and
generate at least one annotation record based on the evaluation of the crowdsourced annotations.

22. A collaborative annotation system, the system comprising:
an electronic processor configured to:
access at least one annotation record, wherein the at least one annotation record is based on crowdsourced annotations obtained for a collaborative annotation project associated with a set of medical images, and
generate training data based on the at least one annotation record.

23. The collaborative annotation system of claim 22, wherein the electronic processor is configured to train a machine learning model using the training data, wherein the machine learning model performs a medical image analysis function.

24. A collaborative annotation system, the system comprising:
an electronic processor configured to:
access training data associated with annotation records based on crowdsourced annotations obtained for a collaborative annotation project associated with a set of medical images, and
develop a model using machine learning using the training data, wherein the model is associated with a medical image analysis function.

25. The collaborative annotation system of claim 24, wherein the electronic processor is configured to:
receive a medical image associated with a patient,
apply the model to the medical image to determine a predicted annotation for the medical image, and
generate an annotated medical image including the predicted annotation for the medical image.

26. A collaborative annotation system, the system comprising:
an electronic processor configured to:
obtain crowdsourced annotations associated with at least one medical image from a dispersed group of annotators,
evaluate the crowdsourced annotations to determine an annotation contribution to the crowdsourced annotations for each annotator included in the dispersed group of annotators, and
generate and associate a digital reward for at least one annotator included in the dispersed group of annotators based on a corresponding annotation contribution for the at least one annotator.
